(19) United States
(12) Patent Application Publication
(10) Pub. No.: US 2024/0316118 A1
(43) Pub. Date: Sep. 26, 2024

(54) ANTIVIRAL NASAL SPRAY FLAVONOIDS?BEE PROPOLIS

(71) Applicants: MCCAFFREY, Stephen, Mark, New South Wales (AU); HAGGART, Ethan Tobias Grey, Hillcrest (NZ); MEYERS, Alicia, Gemma, Eileen, New South Wales (NZ)

(72) Inventor: ALICIA GEMMA EILEEN MEYERS, AUCKLAND (NZ)

(21) Appl. No.: 18/042,597

(22) PCT Filed: Feb. 15, 2022
(86) PCT No.: PCT/AU2022/050103

§ 371 Date: Feb. 22, 2023

(30) Foreign Application Priority Data
Feb. 3, 2022 (AU) 2022200728

(51) Int. Cl. A61K 35/644; A61K 9/00; A61K 31/352; A61K 31/355; A61K 31/375; A61K 33/30; A61K 36/61; A61K 47/36

(52) U.S. Cl. CPC A61K 35/644; A61K 9/0043; A61K 31/352; A61K 31/355; A61K 31/375; A61K 33/30; A61K 36/61; A61K 47/36

(57) ABSTRACT

A nasal spray formulation, a manufacturing method and a use thereof is provided. The nasal spray formulation for the prevention of contracting Coronavirus comprises: a bee pollen, and/or a bee propolis, and/or a honey; the nasal spray formulation is an antiviral nasal spray formulation for inhibiting cell entry of Coronavirus and it's variants, through the nasal passages. Mixtures of different flavonoids or combination of flavonoids with antiviral synthetic drugs provide an enhancement of their antiviral effects. The molecular mechanisms of their antiviral effects mainly consist in the inhibition of viral neuraminidase, proteases and DNA/RNA polymerases, as well as in the modification of various viral proteins. Antioxidants are mainly represented in nature by the liposoluble vitamins E and A, n-carotene, hydro-soluble vitamin C and a wide range of amphipathic molecules, broadly termed phenolic compounds.

These compounds are divided into several sub-classes, including phenolic acids and analogues, stilbenes, flavonoids and their analogues. Flavonoids possess important health protective effects, including anti-inflammatory, anticancer and antiviral properties2 [4,5 3,6,7,8,9,10]. There are in nature more than 6000 flavonoids, which have been structurally identified and divided in classes: flavones (e.g., apigenin), flavanols (e.g., quercetin), flavins (e.g., epigallocatechin-3-gallate), isoflavones (e.g., genistein) and anthocyanidins (e.g., cyanidin).

The invention further provides a use of the said nasal spray formulation in the preparation of medicaments for the prevention of contracting Coronavirus.

In some embodiments, Coronavirus is selected from the group consisting of COVID19, DELTA and OMICRON.

The present invention's technique is scientific and reasonable. As compared with the prior arts, the nasal spray formulation, the manufacturing method and the use thereof according to the present invention have the following advantages:

The only way that we can beat Coronavirus and it's variants, is to prevent the virus entering into our bodies, and to build up our immune system so that the virus has less impact on the infected person. If the virus does not have a host, it cannot survive. This invention will be extremely beneficial for people who are exposed to Coronavirus in their work place, people travelling on public transport, in aircraft, working in air-conditioning offices, during the current pandemic conditions. This invention will make it less important to wear a mask to prevent contracting Coronavirus and its variants.

The inventor has developed the nasal spray formulation that is scientifically proven to be effective for the prevention of contracting COVID19 and DELTA and the Omicron variant's.

This invention incorporates a chitosan based hydrogen and/a nano-chitosan-based hydrogel for the Manuka honey, UMF 20+(MGO), which improves the Dispersity index (PdI), pH value and molecular weight for use in the nasal spray formula.

In the present invention, the water soluble bee propolis, separated phenolic compounds with chromatography separation of the specific flavonoids and phenols in particular an aqueous dispersion of bee propolis, preferably a bee propolis sub-micron aqueous dispersions via a direct ultra-sonication to enhance antiviral properties and mixed with small amounts of the water soluble bee pollen and/Bee propolis to create synergistical enhancement for rapid antiviral action.

### TECHNICAL FIELD

The present invention relates to a spray, particularly a nasal spray formulation as well as a manufacturing method and a use thereof

### BACKGROUND OF THE INVENTION

With the current pandemic of Coronavirus, the most important issue for medical scientists is preventing people from contracting COVID19 and DELTA and OMICRON virus variants.

Masks are currently used to help prevent people becoming infected with Coronavirus or its variants.

The benefits of the vaccinations for Coronavirus variants are short term. For example, the Coronavirus vaccines have a short half-life, thereby necessitating repeated injections or ‘booster shots’ of the vaccination. The virus is growing more resistant to the vaccines, with the more people who get infected with COVID19 after being vaccinated, and then pass on the virus to other vaccinated people.

There are several drugs (e.g., antiviral drugs, antibiotics, corticosteroids, and interferons) are repurposed for COVID-19 treatment without an evidence of efficacy in humans [5,6,38]. Anemia and diarrhea (common causes of malnutrition in COVID-19) are reported adverse effects of antiviral drugs (e.g., ribavirin and hydroxychloroquine) in previous coronavirus infections [38]. COVID-19 patients receiving antibiotics are at high risk for thrombogenic complications, liver damage, malnutrition, and hypoproteinemia [39,40,41]. Therefore, natural agents with multiple bioactivities are being explored for their potential to correct immune deficiency in COVID-19 and subsequently improve disease outcomes.

Flavonoids from propolis and bee honey are exhibiting the highest binding to severe acute respiratory syndromecoronavirus-2 (SARS-CoV-2) proteins and host cell receptor/proteases in silico along with their possible antiCoronavirus Disease 2019 (COVID19) effects.

However, it is worthwhile to note that dietary intake of flavonoids ranges from 5 to 100 mg/day (quercetin [Que] and its glycosides account for about 75%), mostly depending on the consumption of fruits and vegetables and the intake of tea. 10,

Que is largely metabolized in the intestine and liver 12,13 so that its plasma level is normally low. However, after consuming Que-rich foods, the plasma level of this flavonoid increases

Quercetin inhibits the replication of cytomegalovirus inoculated HeLa cells at a half inhibitory concentration (IC50) of 3.2±0.8 μM and with a selectivity index of 22.78 Dengue virus type 2 (DENV-2) replication in Vero cells is inhibited by Que at an IC50 of

35.7 μg/mL, causing a DENV-2 ribonucleic acid (RNA) reduction of 67%. This is attributed to Que's ability to either block virus entry or inhibit viral replication enzymes such as viral polymerases.79 Que and fisetin inhibited DENV-2 and DENV-3 infection in the absence or presence of enhancing antibody (>90%, P<0.001).80 Athletes supplemented with Que are protected from stress-induced susceptibility to upper respiratory tract infection,81 which is not related to immunomodulation.82,83 Que and SARS-COV-2. There is currently no nasal spray formula treatment specifically developed for the prevention of contracting COVID19 and DELTA and OMICRON virus variants. Quercetin has been investigated for its possible antiviral effect on several members of the Coronaviridae family. To the best of our knowledge, one of the first reports exploring the antiviral effect of Que on coronaviruses appeared in 1990 suggesting that it reduced infectivity of human and bovine coronaviruses, OC43 and NCDCV, respectively, by 50% at a concentration of 60 μg/mL.56 On a different Coronaviridae of veterinarian interest, PEDV, quercetin 7-O-rhamnoside inhibited PEDV replication in Vero cells with an IC50 of

0.014 μg/mL and a 50% cytotoxicity concentration (CC50) of 100 μg/mL.113 Luteolin and Que showed the capacity to block the entry of SARS-CoV into host cells.114 Luteolin inhibited, in a dosedependent manner, SARS-CoV infection of Vero E6 cells with a half-effective concentration (EC50) value of 10.6 μM (CC50=155 μM), while Que antagonized HIVluc/SARS pseudo-type virus entry with an EC50 of 83.4 μM.114 Thus, Que offers great promise as a potential drug in the clinical treatment of SARS.114.

SARS coronavirus, described in 2003,115 is a single-stranded RNA virus, which uses ribosome sites to encode 2 replicase glycoproteins, PP1a and PP1b, which mediate viral replication. 115,116 Once these precursor glycoproteins are synthesized, 3C-like protease (3CLpro) plays a critical role in the lytic release of its replicates.117.

Quercetin, identified as one of the constituents of Pichia pastoris displayed good inhibition toward 3CLpro with an IC50 value of 73 M.118 Quercetin-3-O-βgalactoside binds to SARSCov 3 Cl protease and inhibits its proteolytic activity with an IC50 of 42.79

±4.95 μM.119 This inhibitory action on 3CLpro is dependent on the hydroxyl group of Que which, as shown through molecular modeling and Q189A mutation, recognizes Gln189 as a crucial site on 3CLpro responsible for the binding of Que.120 Que was also identified as being able to block SARS coronavirus entry into Vero E6 cells with a half effective concentration (EC50) of 83.4 μM and with low cytotoxicity (CC50 3.32 mM).114 SARS-CoV-2, the virus responsible for the coronavirus disease 2019 (COVID-19) pandemic,120 belongs to the genus Betacoronavirus and subgenus Sarbecovirus and, due to its similar receptor-binding domain, it is assumed, similarly to SARS-CoV, to infect type II pneumocytes entering via the angiotensin-converting enzyme-2 (ACE2) receptor.121 SARSCov-2 protease 3 Cl maintains the same Gln189 site122 of SARS-Cov 3CLpro, which previously was identified as the binding site for the hydroxyl groups of Que and its derivatives.

Considering bioavailability issues, a nasal spray of dilute Que has been suggested to be a suitable vehicle, administered regularly at low doses during the early stages of infection, as it could attenuate entry of the virus into cells and so halt progress of the infection, possibly leading to a reduced need for hospitalization.

Quercetin: Antiviral Significance and Possible COVID-19 Integrative Considerations

Pawan K. Agrawal https://orcid.org/0000-0002-7149-83581, Chandan Agrawall, and Gerald Blunden2 Propolis, Bee Honey, and Their Components Protect against Coronavirus Disease 2019 (COVID-19): A Review of In Silico, In Vitro, and Clinical Studies by Amira Mohammed Ali 1,2,* and Hiroshi Kunugi.

Intranasal administration of the nasal spray formulations (Examples 1 to 5) that provides good coverage of the nasal passages, is effective for the prevention of contracting COVID19 and DELTA and OMICRON, variants.

With frequent use, the biochemical properties of the bee propolis and bee pollen and Manuka honey will assist the body to develop a stronger immunity to viruses, it has also been found that Que binds better to Spike protein, ACE2, RdRp, and PLpro, indicating good potential against SARSCoV-2.124 SARS-CoV 3CLpro shares some features with SARS-CoV2; therefore, Que might exert some protective or curative role against COVID-19, particularly considering its antioxidant and antiinflammatory36properties, as well as the effects of Que observed against other viruses described above. It also modulates the cellular unfolded protein response (UPR). As coronaviruses can utilize the UPR to complete their entire replication cycle, Que may have anticoronavirus effects through its modulation of this pathway. Coronavirus appears to be susceptible to the inhibitory actions of zinc, which may prevent viral entry into cells.

### STATEMENT OF THE INVENTION

To overcome at least one of the above deficiencies in the prior art, the present invention provides a nasal spray formulation which is an antiviral nasal spray formulation for inhibiting cell entry of Coronavirus and it's variants through the nasal passages, as well as a manufacturing method and a use thereof.

To achieve the above purposes, the invention provides a nasal spray formulation for the prevention of contracting Coronavirus comprising at least one active ingredient selected from a group consisting of a water soluble bee pollen, a water soluble bee propolis, and a honey, preferably Manuka honey, more preferably Manuka honey with a UMF of 20+.

In the invention, the term “UMF” stands for Unique Manuka Factor, which is a measurement of the antimicrobial and antibacterial properties in Manuka honey, also referred to as MGO aka methylgloxal. MGO, or methylgloxal, refers to the well-documented antibacterial and antimicrobial ingredient in Manuka honey. 850 MGO Manuka honey would have a 20+ UMF.

In some embodiments, the nasal spray formulation comprises the propolis and the honey.

In some embodiments, the nasal spray formulation comprises the bee pollen, the beepropolis and the honey.

In the present invention, the bee propolis is extracted by a method that maintains the bioactive ingredients, e.g., phenolic compounds including flavonoids such as Quercetin-3-O-Glucoside, Quercetin-7-O-rhamnoside and Quercetin-3-B-galactoside. The bee propolis is also tested for its biochemical properties.

In some embodiments, e.g, the bee propolis which has high flavonoids, phenolic contents showing antiviral properties is extracted using an extraction process that maintains the potency of the naturally occurring chemicals found in the bee propolis, and in some preferable embodiments, the extraction process is selected from a group consisting of an aqueous extraction or ethanol extraction process, and a vacuum resistive heating extraction (VRHE) process.

In some embodiments, the bee propolis may be enhanced by utilizing a process of ultrasonication. For example, a bee propolis sub-micron aqueous dispersion is used. The bee propolis sub-micron aqueous dispersion can be obtained through a conventional method in the art, e.g., a (direct) ultra-sonication. Ultra sonication is also utilized to enhance the bioactive ingredients.

In one embodiment, the bee propolis contains approximately 21% of Quercetin, for example, approximately 7% by weight of each of the afore mentioned Quercetin Glucosides. These Quercetin can be obtained using a chromatographic separation. For example, the bee propolis is obtained by an isocratic elution at 25° C., and further enhancement is achieved by a ultra-sonication, for approximately 20-30 minute. It is believed that such an treatment can achieve a synergistic effect.

In some embodiments, the nasal spray formulation further comprises of a chitosan-based hydrogel.

In a particular embodiment, the nasal spray formulation further comprises a chitosan-based hydrogel (e.g., a nano-chitosan-based hydrogel and the Manuka honey, and in another particular embodiment, the nasal spray formulation further comprises a 1nano-chitosan-based hydrogel and the Manuka honey with a UMF of 20+.

In some embodiments, the at least one active ingredient is present in an amount from approximately 21% of Quercetin, made up of 7% of each of the afore mentioned Quercetin Glucosides by weight, obtained from Chromatographic separation. This part of the active ingredient is obtained by isocratic elution at 25° C., and further enhancement is achieved by ultra sonication, of 12% of the active ingredient for approximately 20-30 minutes, to achieve a synergistic effect.

In some embodiments, the water soluble bee pollen is present in an amount from approximately 3% to 8% by weight; and/or the water soluble bee propolis is present in an amount from 0.1% to 0.8% by weight, preferably from approximately 0.1% to 0.5% by weight, and more preferably 0.3% to 0.4% by weight; and/or the honey is present in an amount from approximately 0.4% to 0.7% by weight, preferably 0.5% by weight.

In some embodiments, the nasal spray formulation comprises at least one additional bioactive substance.

In some embodiments, the additional bioactive substance is one or more antioxidants and/or one or more phytochemicals and/or one or more amino acids and/or one or more proteins and/or one or more lipids.

In some embodiments, the phytochemicals are flavonoids; wherein the flavonoids are preferably selected from the group consisting of flavonoids flavone, isoflavone, flavan, isoflavan, flavanone, flavonol, flavan-3-ol, dihydroflavonol, flavanonol, anthocyanidin, proanthocyanidin, aurone, chalcone, dihydrochalcone, flavonolignans, and derivatives thereof; more preferably flavonoids, luteolin, rutin, quercetin, naringenin, CAPE, apigenin, vitexin, and combinations thereof.

The flavone preferably is selected from the group consisting of luteolin and Rutin. Approximately 0.5% of zinc may be added into the flavone.

The isoflavone preferably is selected from the group consisting of daidzein, genistein and formononetin.

The flavan preferably is epigallocatechin-3-gallate. The isoflavan preferably is glabridin or licoricidin.

The flavanone preferably is eriodictyol.

The flavonol preferably is selected from the group consisting of rutin, myricetin, kaempferol, gossypetin and quercetin.

The flavan-3-ol preferably is selected from the group consisting of catechin, theaflavin, gallocatechin, catechin gallate, gallocatechin gallate, epicatechin, epigallocatechin, epigallocatechin gallate and epigallocatechin gallate.

The dihydroflavonol preferably is selected from the group consisting of naringenin, taxifolin and aromadendrin.

The anthocyanidin preferably is selected from the group consisting of cyanidin, delphinidin, pelargonidin and maividin.

The aurone preferably is sulphuretin.

The chalcone preferably is 2′-hydroxy-4-methoxy-chalcone.

The dihydrochalcone preferably is phloretin or phloridzin.

The flavonolignan preferably is silibinin or silichristin.

In some embodiments, the antioxidants are one or more of a vitamin or vitamin derivative, preferably the antioxidants are selected from the group consisting of vitamin C, β-carotene, vitamin A, vitamin A ester, vitamin E, vitamin E ester, and phenolic compounds, more preferably vitamins C and E. In some embodiments, the antioxidants are present in an amount of from 0.1% to 0.6% by weight.

In some embodiments, the phytochemicals are all the plant material. Especially, from 10% to 80% by weight, preferably from 20% to 70% by weight of the phytochemicalsare from the bee propolis, bee pollen and Manuka honey.

In some embodiments, the nasal spray formulation further comprises a solvent, preferably the solvent is water or ethanol, more preferably the solvent is purified water.

In some embodiments, the amount of the active ingredient can be determined depending on the strength of the bioactive molecules. In one embodiment, the nasal spray formulation comprises approximately from 0.5% to 0.10% by weight of bee propolis, 0.4% to 0.7% by weight, preferably 0.5% by weight of Manuka honey with a UMF of 20+, about 0.3% to 0.4% by weight of vitamin C (absorbic acid), and an able liquid carrier.

In one embodiment, the able liquid carrier is the solvent.

This invention uses a combination of naturally occurring chemicals and synthetic chemicals.

The invention further provides a method for manufacturing said nasal spray formulation comprising a bee pollen and/or a bee propolis and/or a honey is mixed with a solvent; preferably the bee propolis is diluted with the solvent to provide a liquid mixture, and the honey with a UMF of 20+ is added to the liquid mixture; more preferably the bee propolis is diluted with the solvent and at least one additional bioactive substance to provide a liquid mixture, and the honey with a UMF of 20+ is added to the liquid mixture.

In some embodiments, the bee propolis is diluted with purified water or alcohol, and extra vitamins C, E are added to provide the liquid mixture, and Manuka honey with a UMF of 20+ is added to the liquid mixture.

### DETAILED DESCRIPTION OF THE INVENTION

Herein below the preferred examples of the present invention are illustrated in combination with the drawings. It shall be understood that the preferred examples described herein are only used for illustrating and explaining the present invention, but shall in no way be construed as restricting the present invention.

There is no effective antiviral nasal spray formulation for the prevention of contracting coronavirus currently on the market.

This is an ideal opportunity for a pharmaceutical manufacturing company to provide the most effective prevention treatment option available, an antiviral nasal spray formulation, that will help to improve the body's natural immune system, and can be used as an effective treatment to minimize severity of symptoms, reduce hospitalization and mortality rates.

There are several drugs (e.g., antiviral drugs, antibiotics, corticosteroids, and interferons) are repurposed for COVID-19 treatment without an evidence of efficacy in humans [5,6,38]. Anemia and diarrhea (common causes of malnutrition in COVID-19) are reported adverse effects of antiviral drugs (e.g., ribavirin and hydroxychloroquine) in previous coronavirus infections [38]. COVID-19 patients receiving antibiotics are at high risk for thrombogenic complications, liver damage, malnutrition, and hypoproteinemia [39,40,41]. Therefore, natural agents with multiple bioactivities are being explored for their potential to correct immune deficiency in COVID-19 and subsequently improve disease outcomes.

The present invention provides a nasal spray formulation for the prevention of contracting Coronavirus comprising at least one active ingredient selected from a group consisting of a bee pollen, a bee propolis, and a honey, preferably Manuka honey, more preferably Manuka honey with a UMF of 20+.

In some embodiments, the nasal spray formulation comprises the propolis and the honey.

In some embodiments, the nasal spray formulation comprises the bee pollen, the beepropolis and the honey.

In the present invention, the bee propolis is extracted by a method that maintains the bioactive ingredients, particularly Quercetin. The bee propolis is tested for it's biochemical properties.

In some embodiments, the bee propolis that has high flavonoids, phenolic contents showing antiviral properties is extracted using an extraction process that maintains the potency of the naturally occurring chemicals found in the bee propolis, and in some preferable embodiments, the extraction process is selected from a group consisting of aqueous extraction or ethanol extraction process, and vacuum resistive heating extraction (VRHE) process.

In some embodiments, the bee propolis is an aqueous dispersion of bee propolis, preferably bee propolis sub-micron aqueous dispersions. The bee propolis sub-micron aqueous dispersion can be obtained through conventional method in the art, e.g., a direct ultra sonication.

Clinical trials and studies have shown that flavonoids in the bee propolis inhibit the proteolytic processing of s protein by host proteases.

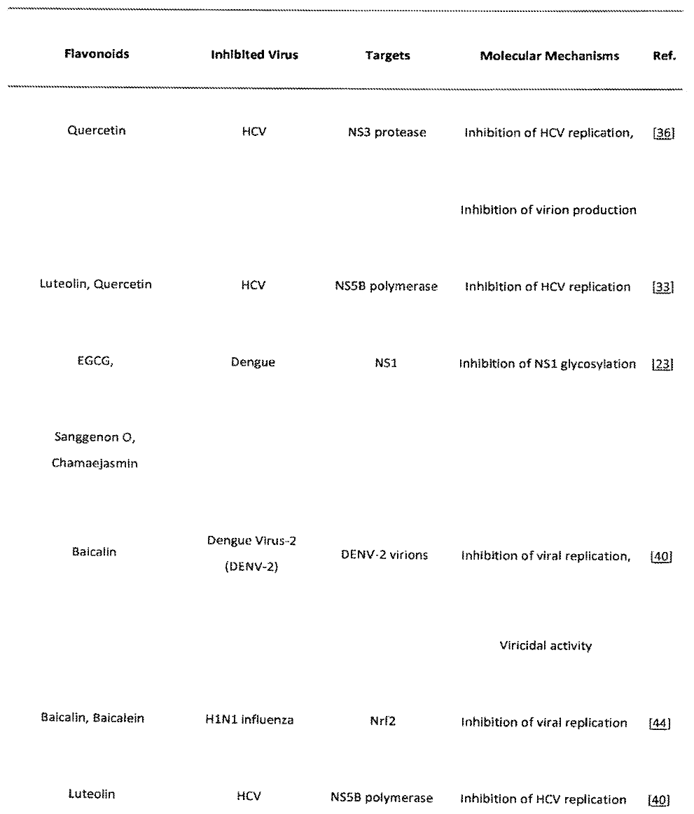

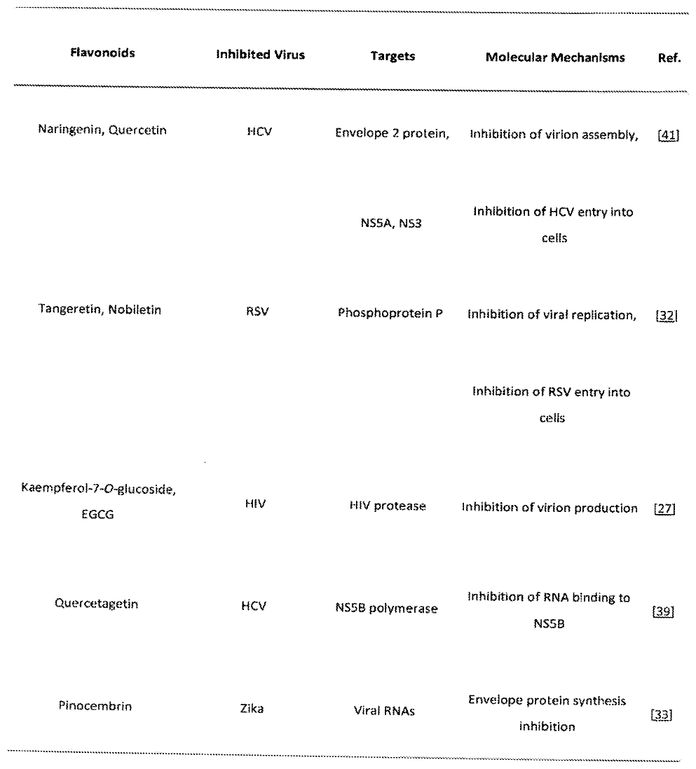

Flavonoids in the bee propolis may inhibit the binding of SARS-CoV-2 to host cell receptors ACE-II and is a type I integral membrane protein that functions as a metalloprotease enzyme. It contains 805 amino acids comprising one HEXXH-E zinc-binding consensus sequence in its active sites [48]. It is suggested that COVID-19 is highly contagious because SARS-CoV-2 easily enters human cells by binding human ACE-II more strongly than other coronaviruses [82-84]. The binding of S protein to ACE-1l does not disrupt the ACE. FIG. 3 shows chemical structures of flavonoids with the highest binding affinity to target proteins of severe acute respiratory syndrome-coronavirus (SARS-CoV-2).

In some embodiments, the nasal spray formulation comprises a composition of the honey and a nano-chitosan-based hydrogel, preferably a nano-chitosan-based hydrogel for the Manuka honey, more preferably a nano-chitosan-based hydrogel for the Manuka honey with a UMF of 20+.

In some embodiments, the water soluble bee pollen is present in an amount from 3% to 8% by weight; and/or the bee propolis is present in an amount from 0.1% to 0.8% by weight, preferably from 0.1% to 0.5% by weight, preferably 0.3% to 0.4% by weight; and/or the honey is present in an amount from approximately 0.4% to 0.7% by weight, preferably 0.5% by weight.

In some embodiments, the nasal spray formulation further comprises at least one additional bioactive substance.

In some embodiments, the additional bioactive substance is one or more antioxidants and/or one or more phytochemicals and/or one or more amino acids and/or one or more proteins and/or one or more lipids, preferably one or more antioxidants and/or one or more phytochemicals, more preferably one or more antioxidants.

In some embodiments, the phytochemicals are flavonoids; wherein the flavonoids are preferably selected from the group consisting of flavonoids flavone, isoflavone, flavan, isoflavan, flavanone, flavonol, flavan-3-ol, dihydroflavonol, flavanonol, anthocyanidin, proanthocyanidin, aurone, chalcone, dihydrochalcone, flavonolignans, and derivatives thereof; more preferably flavones, apigenin, vitexin, quercetin, rutin, naringenin, and combinations thereof.

The flavone preferably is selected from the group consisting of luteolin and chrysin. Approximately 0.5% of zinc may be added into the flavone.

The isoflavone preferably is selected from the group consisting of daidzein, genistein and formononetin.

The flavan preferably is epigallocatechin-3-gallate. The isoflavan preferably is glabridin or licoricidin.

The flavanone preferably is eriodictyol.

The flavonol preferably is selected from the group consisting of, rutin, myricetin, kaempferol, gossypetin and quercetin.

The flavan-3-ol preferably is selected from the group consisting of catechin, theaflavin, gallocatechin, catechin gallate, gallocatechin gallate, epicatechin, epigallocatechin, epigallocatechin gallate and epigallocatechin gallate.

The dihydroflavonol preferably is selected from the group consisting of naringenin, taxifolin and aromadendrin.

The anthocyanidin preferably is selected from the group consisting of cyanidin, delphinidin, pelargonidin and malvidin.

The aurone preferably is sulphuretin.

The chalcone preferably is 2′-hydroxy-4-methoxy-chalcone.

The dihydrochalcone preferably is phloretin or phloridzin.

The flavonolignan preferably is silibinin or silichristin

Flavonoids and their analogues possess important health protective effects, including antiinflammatory, anticancer and antiviral properties. There are in nature more than 6000 flavonoids. Flavonoids occur in their free or conjugated form or are often esterified with one or two sugars with O-glycosidic or C-glycosidic bonds. Flavonoids antiviral activities are listed in Tables 1-4.

In some embodiments, the antioxidants are one or more of a vitamin or vitamin derivative, preferably the antioxidants are selected from the group consisting of vitamin C, β-carotene, vitamin A, vitamin A ester, vitamin E, vitamin E ester, and phenolic compounds, more preferably vitamins C and E.

For example, the vitamin C is hydro-soluble vitamin C with added zinc, and the vitamins E and A are liposoluble vitamins E and A.

In some embodiments, the phytochemicals are all the plant material. Especially, 10% to 80% by weight, preferably from 20% to 70% by weight of the phytochemicals are from the bee propolis, bee pollen and Manuka honey.

In some embodiments, the nasal spray formulation further comprises a solvent, preferably the solvent is water or ethanol, more preferably the solvent is purified water.

In some embodiments, the amount of the active ingredient can be determined depending on the strength of the bioactive molecules. In one embodiment, the nasal spray formulation comprises approximately from % 0.5 to 0.10% by weight of bee propolis, 0.4% to 0.7% by weight, preferably 0.5% by weight of Manuka honey with a UMF of 20+, about 0.3% to 0.4% by weight of vitamin C (absorbic acid), and an able liquid carrier; wherein the able liquid carrier is the solvent.

This invention may use a combination of naturally occurring chemicals and synthetic chemicals.

The invention further provides a method for manufacturing the said antiviral nasal spray formulation, comprising: a water soluble bee pollen and/or a water soluble bee propolis and/or a honey. The solvent used in the art of the extraction of the crude propolis is required be suitable for the intranasal application of use.

In some embodiments, the bee propolis is diluted with the solvent to provide a liquid mixture, and the honey with a UMF of 20+ is added to the liquid mixture, preferably the bee propolis is diluted with the solvent and at least one additional bioactive substance is added to provide a liquid mixture, and the honey with a UMF of 20+ is added to the liquid mixture.

In some embodiments, the bee propolis is diluted with purified water or alcohol, and extra vitamins C, E are added to provide the liquid mixture, and Manuka honey with a UMF of 20+ is added to the liquid mixture.

The invention further provides a use of said nasal spray formulation in the preparation of medicaments for the prevention of contracting Coronavirus.

In some embodiments, Coronavirus is selected from the group consisting of COVID19, DELTA and OMICRON.

In some embodiments, the bee propolis is extracted propolis such as Green or Brown propolis and the flavonoids are extracted flavonoids including Quercetin and/or Rutin, obtained through the chromatographic separation. Enhancement of the antiviral properties is achieved via a direct ultrasonication. The combination of water soluble bee propolis and bee pollen, and the Quercetin and Rutin creates a synergistic effect.

In the invention, the nasal spray formulation is a antiviral nasal spray formulation and may include honey, preferably Manuka honey UMF 20+, together with absorbic acid, suspended in Chitosan (Mushroom Chitosan Oligosaccharide COS) based hydrogel and the able liquid carrier of purified water, prepared for intranasal administration.

The nasal spray formulation for the prevention of contracting Coronavirus is highly effective and beneficial for most people, except those who are allergic to bee products.

The synthetic derivatives of the key ingredients from the bee propolis and bee pollen can be used to produce an effective antiviral nasal spray formulation, that does not cause any allergic reactions in those who have allergies that can cause Anaphylaxis.

The concentrations of ingredients of nasal spray formulation varies depending upon the levels of the Quercetin and other active ingredients found in the bee propolis and/bee pollen.

This invention may use a combination of naturally occurring chemicals and synthetic chemicals.

Key ingredients including high levels of: Quercetin—CHEBI:16243—quercetin; 2-(3,4-dihydroxyphenyl)-3,5,7-trihydroxy-4H-1 benzopyran-4-one, ChEBI; 3,3′,4′,5,7-pentahydroxyflavone, ChEBI; 3, 5, 7, 3′ Chemical C15H10O7

Quercetin is naturally found in bee propolis, in high levels that is sourced from regions with a particular botanical flora.

Quercetin, a naturally occurring dietary flavonoid, is well known to ameliorate chronic diseases and aging processes in humans, and its antiviral properties have been investigated in numerous studies. In silico and in vitro studies demonstrated that quercetin can interfere with various stages of the coronavirus entry and replication cycle such as PLpro, 3CLpro, and NTPase/helicase.

Due to its pleiotropic activities and lack of systemic toxicity, quercetin and its derivatives may represent target compounds to be tested in future clinical trials to enrich the drug arsenal against coronavirus infections. There is evidence that quercetin in combination with, for example, vitamins C and D, may exert a synergistic antiviral action that may provide either an alternative or additional therapeutic/preventive option due to overlapping antiviral and immunomodulatory properties. This review summarizes the antiviral significance of quercetin and proposes a possible strategy for the effective utilization of natural polyphenols in our daily diet for the prevention of viral infection.

Pinocembrin (5,7-dihydroxyflavanone) is one of the primary flavonoids isolated from bee propolis and a variety of plants, mainly from Pinus heartwood, Eucalyptus, Populus, Euphorbia, and Sparattosperma leucanthum, in the diverse flora and purified by various chromatographic techniques. Pinocembrin is a major flavonoid molecule incorporated as multifunctional in the pharmaceutical industry. Its vast range of pharmacological activities has been well researched including antimicrobial, anti-inflammatory, antioxidant, and anticancer activities. In addition, pinocembrin can be used as neuroprotective against cerebral ischemic injury with a wide therapeutic time window, which may be attributed to its antiexcitotoxic effects. Pinocembrin exhibits pharmacological effects on almost all systems, and Flavonoid compounds in general and in particular pinocembrin are well-known plant compounds that have antimicrobial and anti-inflammatory properties. Scientists and clinicians have demonstrated in vitro and in vivo the biological or pharmacological properties of pinocembrin and have elucidated mechanisms of action, including an inhibiting factors that prevent a virus from entering into the membrane and inhibiting cell proliferation of the virus.

This invention for a nasal spray formulation using naturally occurring chemicals, and/or their synthetic derivatives, to produce the bioactive molecules found in bee propolis and bee pollen, that provide antiviral properties for the prevention of contracting Coronavirus, (COVID19 AND DELTA AND OMICRON VARIANT'S).

Some biochemical reactions of flavonoids are altered by ingestion, that is why this invention for a nasal spray formulation is particularly effective for the prevention of contracting Coronavirus and it's variant's.

Mixtures of different flavonoids or combination of flavonoids with antiviral synthetic drugs provide an enhancement of their antiviral effects. The molecular mechanisms of their antiviral effects mainly consist in the inhibition of viral neuraminidase, proteases and DNA/RNA polymerases, as well as in the modification of various viral proteins.

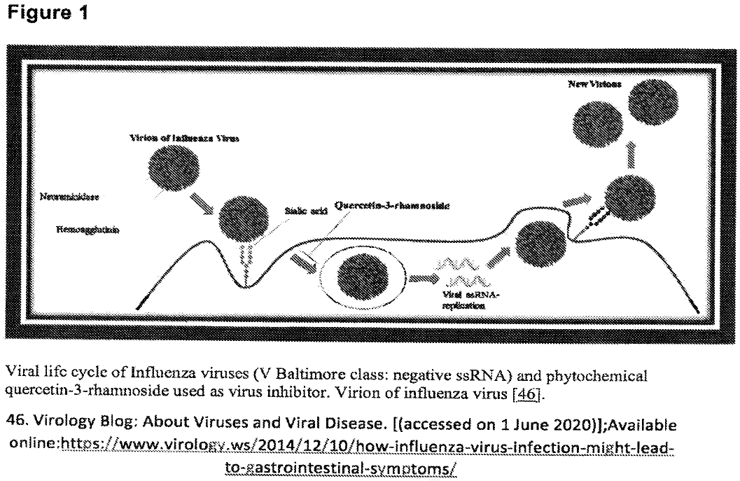

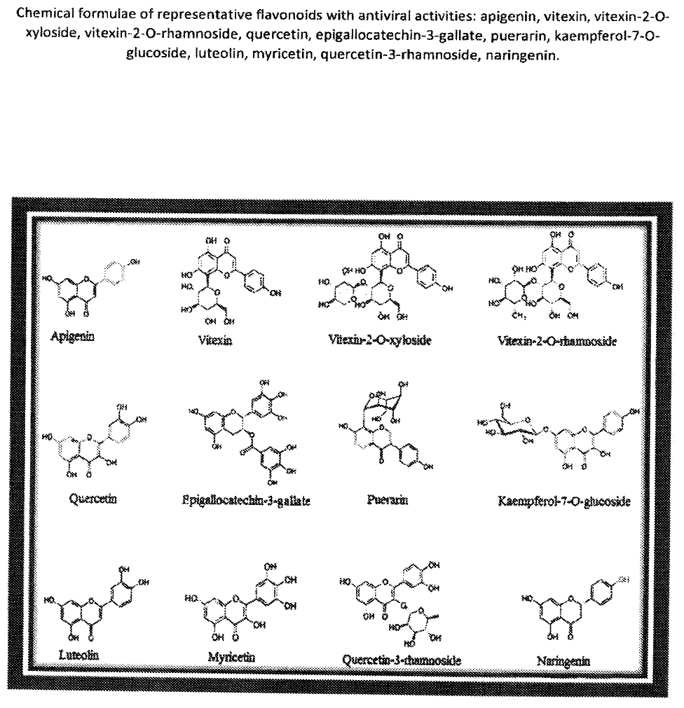

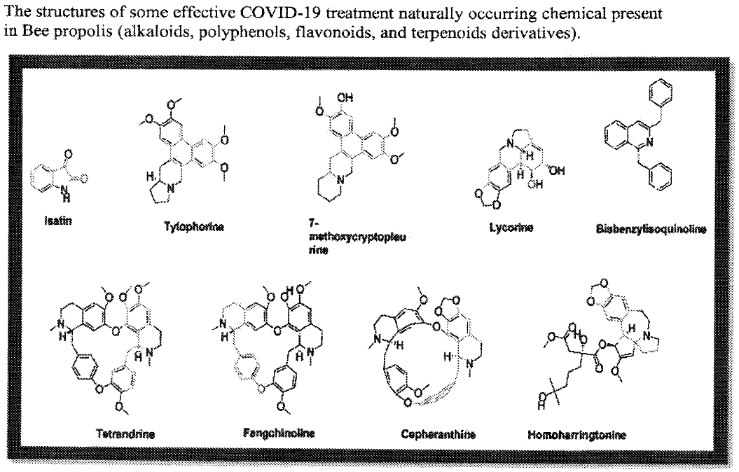

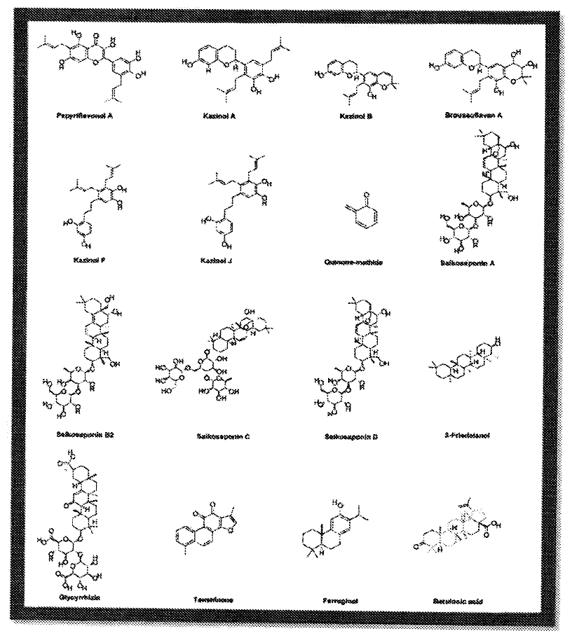

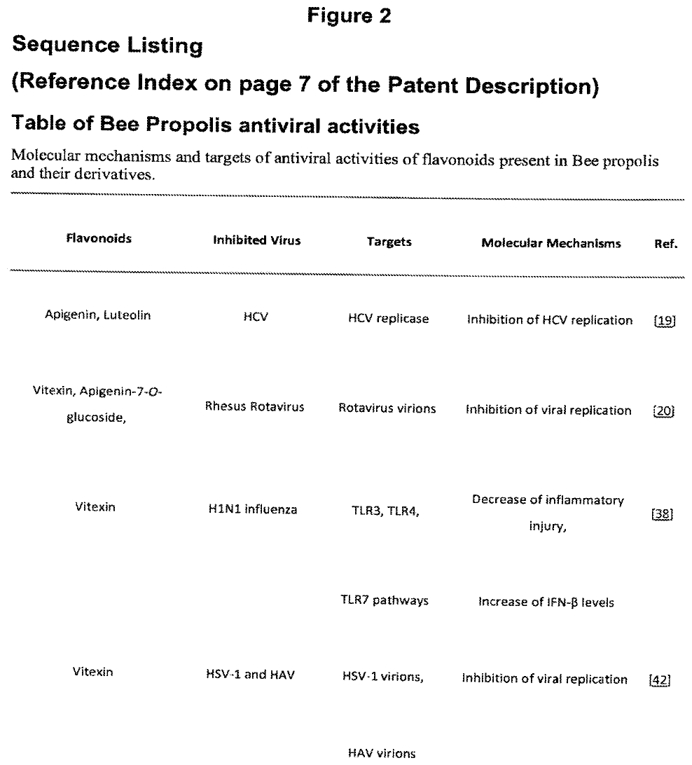

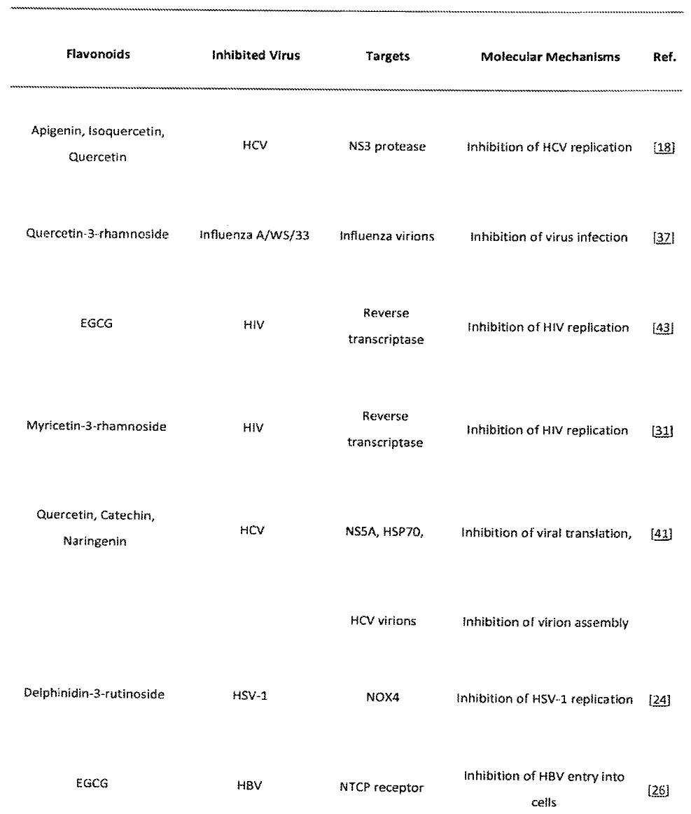

FIG. 1 shows Chemical structure of quercetin. FIG. 2 shows viral life cycle of Influenza viruses (V Baltimore class: negative ssRNA) and phytochemical quercetin-3-rhamnoside used as virus inhibitor. Virion of influenza virus [46].

Virology Blog: About Viruses and Viral Disease. [(accessed on 1 Jun. 2020)]; Available online: https://www.virology.ws/2014/12/10/how-influenza-virus-infection-might-lead-togastrointestinal-symptoms/

Lastly, it should be noted that the above stated examples are only preferable examples of the present invention and shall not be construed as limitation of the present invention. Though the present invention is illustrated with reference to the preceding Examples in details, persons skilled in the art can still make amendments to the technical solutions as disclosed in the those examples, or make equivalent replacement to part of the technical solutions therein. Any amendment, equivalent replacement or improvement under the spirit and principle of the present invention shall all be included in the protection scope of the present invention.

### Example 1

A nasal spray formulation for the prevention of contracting Coronavirus consists of: Water soluble bee propolis 0.1% (w/w); Manuka honey with a UMF of 20+0.5% (w/w); vitamin C (absorbic acid) 0.3% (w/w); vitamin E 0.3% (w/w); and zinc 0.03% (w/w) with the addition of the Quercetin and the flavonoids that have been enhanced by ultrasonication,
a balance of purified water; wherein the purified water is a solvent; the vitamin C is hydro-soluble vitamin C; the vitamin E is liposoluble vitamin E.

The method for manufacturing said nasal spray formulation comprising: the bee propolis was diluted with the purified water, and extra vitamins C, E were added to provide the liquid mixture, and the Manuka honey with a UMF of 20+ was added to the liquid mixture, and suspended in a chitosan based hydrogel of 21%.

### Example 2

The nasal spray formulation and process for preparing the same of Example 2 are the same as that of Example 1, except the bee pollen is present in an amount of 0.5% (w/w).

### Example 3

The nasal spray formulation and process for preparing the same of Example 3 are the same as that of Example 1, except the solvent is ethanol.

### Example 4

A nasal spray formulation for the prevention of contracting Coronavirus consists of: Water soluble bee pollen 4% (w/w); Water soluble bee propolis 0.1%
(w/w); Manuka honey with a UMF of 20+0.5% (w/w); vitamin C (absorbic acid) 0.3% (w/w); vitamin E 0.2%; and a balance of purified water; wherein the purified water is a solvent the vitamin C is hydro-soluble vitamin C; the vitamin E is liposoluble vitamin E.

The method for manufacturing said nasal spray formulation of comprising the water soluble bee pollen and the water soluble bee propolis were diluted with the purified water, and extra vitamins C, E and zinc were added to provide the liquid mixture, and the Manuka honey with a UMF of 20+, and nano-chitosan hydrogel were added to the liquid mixture.

### Example 5

A nasal spray formulation for the prevention of contracting Coronavirus consists of: bee propolis 0.1% (w/w); Manuka honey with a UMF of 20+
0.5% (w/w); vitamin C (absorbic acid) 0.4% (w/w); Chitosan (Mushroom Chitosan Oligosaccharide COS) 20% (w/w); and a balance of purified water; wherein the purified water is a solvent; the vitamin C is hydro-soluble vitamin C.

The method for manufacturing said nasal spray formulation of comprising the bee propolis was diluted with the purified water, and extra vitamins C was added to provide the liquid mixture, and the Manuka honey with a UMF of 20+ and Chitosan were added to the liquid mixture.

The bee propolis and the Manuka honey UMF 20+, together with absorbic acid, suspended in Chitosan (Mushroom Chitosan Oligosaccharide COS) based hydrogel, and the able liquid carrier of purified water were prepared for intranasal administration.

### FIG. 2

Table of Bee Propolis antiviral activities
Molecular mechanisms and targets of antiviral activities of flavonoids present in Bee propolis andtheir derivatives.

| Flavonoids | Inhibited Virus | Targets | Molecular Mechanisms | Ref. |
|---|---|---|---|---|
| Apigenin, Luteolin | HCV | HCV | Inhibition HCV | [19] |
| Vitexin, Apigenin-7- | | replicase | replication | |
| O-glucoside, | | | | |
| | Rhesus Rotavirus | Rotavirus | Inhibition viral | [20] |
| | | virions | replication | |
| Vitexin | H1N1 influenza | TLR3, | Decrease of | [38] |
| | | TLR4, | inflammatoryinjury, | |
| | | TLR7 | Increase of IFN-β levels | |
| | | pathways | | |
| Vitexin | HSV-1 and HAV | HSV-1 | Inhibition of viral | [42] |
| | | virions, | replication | |
| | | HAV virions | | |
| Apigenin, Isoquercetin, | HCV | NS3 protease | Inhibition of HCV | |
| Quercetin | | | replication | |
| Quercetin-3-rhamnoside | Influenza A/WS/33 | Influenza virions | Inhibition of virus infection | [37] |
| EGCG | HIV | Reverse | Inhibition of HIV | [43] |
| | | transcriptase | replication | |
| Myricetin-3-rhamnoside | HIV | Reverse | Inhibition of HIV | [31] |
| Quercetin, Catechin, | | transcriptase | replication | |
| Naringenin | HCV | NS5A, HSP70, | Inhibition Of viral | [41] |
| | | | translation, | |
| | | HCV virions | Inhibition of virion | |
| | | | assembly | |
| Delphinidin-3-rutinoside | HSV-1 | NOX4 | Inhibition of HSV-1 | [24] |
| | | | replication | |
| EGCG | HBV | NTCP receptor | Inhibition of HBV entryinto | |
| | | | cells | |
| Quercetin | HCV NS3 protease Inhibition of HCV replication, | [ | | |
| Inhibition of virion production | | | | |
| Luteolin, Quercetin | HCV NS5B polymerase | HCV | | |
| | Inhibition of HCV | | | |
| | replication [33] | | | |
| EGCG, | Dengue NS1 | [23] | | |
| | Inhibition of NS1 glycosylation | | | |
| sanggenon O, Chamaejasmin Dengue | Virus-2 | [40] | | |
| Baicalin | DENV-2 virions | | | |
| | Inhibition of viral replication, | | | |
| (DENV-2) | Viricidal activity | | | |

Baicalin, Baicalein H1N1 influenza Nrf2 Inhibition of viral replication [44]
Luteolin HCV NS5B polymerase Inhibition of HCV replication [40]

| Flavonoids | Inhibited Virus Molecular Mechanisms | Targets Ref. |
|---|---|---|
| Naringenin, Quercetin | | |
| HCV | Envelope | 2 |
| protein, | Inhibition of virion assembly, | |
| [41] | | |
| Tangeretin, Nobiletin | RSV | |
| | Phosphoprotein P | |
| | Inhibition of viral replication, | |
| Inhibition of RSV entry into cells | | [32] |
| Kaempferol-7-O-glucoside, | | |
| EGCG | | |
| HIV | HIV protease | [27] |
| | Inhibition virion production | |
| Quercetagetin | | |
| HCV | | |
| NS5B polymerase | | |
| Inhibition of RNA binding to NS5B | | |
| [39] | | |
| Pinocembrin | | |
| Zika | | |
| Viral RNAs | Envelope protein synthesis | |
| inhibition | | |
| [33] | | |
| NS5A, NS3 | Inhibition of HCV entry into cells | 36] |

### REFERENCE INDEX FOR ‘FIG. 2’

19. Manvar D., Mishra M., Kumar S., Pandey V. N. Identification and evaluation of anti hepatitis C virus phytochemicals from Eclipta alba. J. Ethnopharmacol. 2012; 144:545-554. doi: 10.1016/j.jep.2012.09.036
20. Knipping K., Garssen J., van't Land B. An evaluation of the inhibitory effects against rotavirus infection of edible plant extracts. Virol. J. 2012; 9:137-144. doi: 10.1186/1743-422X-9-137.
38. Shi D., Chen M., Liu L., Wang Q., Liu S., Wang L., Wang R. Antiinfluenza A virus mechanism of three representative compounds from Flos Trollii via TLRs signaling pathways. J. Ethnopharmacol. 2020; 253:112634. doi: 10.1016/j.jep.2020.112634.
42. Fahmy N. M., AI-Sayed F., Moghannem S., Azam F., EI-Shazly M., Singab A. N. Breaking Down the Barriers to a Natural Antiviral Agent:
   Antiviral Activity and Moleculai-Docking of Erythrina speciosa Extract, Fractions, and the Major Compound. Chem. Biodivers. 2020; 17:e1900511. doi: 10.1002/cbdv.201900511.
18. Simoes L. R., Maciel G. M., Brandao G. C., Kroon E. G., Castilho R. O., Oliveira A. B. Antiviral activity of Disticella elongata (Vahl) Urb. (Bignoniaceae), a potentially useful source of antidengue drugs from the state of Minas Gerais, Brazil. Lett. Appl. Microbiol. 2011; 53:602-607. doi: 10.1111/j.1472-765X.2011.03146.x.
37. Choi H. J., Song J. H., Park K. S., Kwon D. H. Inhibitory effects of quercetin 3-rhamnoside on influenza A virus replication. Eur. J. Pharm. Sci. 2009; 37:329-333. doi: 10.10t6/j.ejps.2009.03.002.
43. Li S., Hattori T., Kodama E. N. Epigallocatechin gallate inhibits the HIV reverse transcription step. Antivir. Chem. Chemother. 2011; 4:239-243. doi: 10.3851/IMP1774.
31. Ortega J. T., Suarez A. I., Serrano M. L., Baptista J., Pujol F. H., Rangel H. R. The role of the glycosyl moiety of myricetin derivatives in anti-HIV-1 activity in vitro. AIDS Res. Ther. 2017; 14:57-63. doi: 10.1186/s12981-017-0183-6.
41. Khachatoorian R., Arumugaswani V., Raychauduri S., Yeh G. K., Maloney E. M., Wang J., Dasgupta A., French S. W. Divergent antiviral effects of bioflavonoids on the hepatitis C virus life cycle. Virology. 2012; 433:346-355, doi: 10.1016/j.virol.2012.08.029.
24. Di Sotto A., Di Giacomo S., Amatore D., Locatelli M., Vitalone A., Toniolo C., Rotino G. L., Lo Scalzo R., Palamara A. T., Marcocci M. E., et al. A Polyphenol Rich Extract from Solanum melongena L. DR2 Peel Exhibits Antioxidant Properties and Anti-Herpes Simplex Virus Type I Activity In Vitro. Molecules. 2018; 23:2066. doi: 10.3390/molecules23082066.
26. Huang H. C., Tao M. H., Hung F. M., Chen J. C., Lin Z. J., Huang C. (−)-Epigallocatechin-3-gallate inhibits entry of hepatitis B virus into hepatocytes. Antivir. Res. 2014; 111:100-111. doi: 10.1016/j.antiviral.2014.09.009.
36. Bachmetov L., Gal-Tanamy M., Shapira A., Vorobeychik M., Giterman-Galam T., Sathiyamoorthy P., Golan-Goldhirsh A., Benhar I., Tur-Kaspa R., Zemel R. Suppression of hepatitis C virus by the flavonoid quercetin is mediated by inhibition of NS3 protease activity. J. Viral Hepat. 2012; 19:81-88. doi: 10.1111/j.13652893.2011.01507.x.
33. Lee L. J., Loe M. W., Lee R. C., Chu J. J. Antiviral activity of pinocembrin against Zika virus replication. Antivir. Res. 2019; 167:13-24. doi: 10.1016/j.antiviral.2019.04.003.
23. Qamar M. T., Mumtaz A., Naseem R., Ali A., Fatima T., Jabbar T., Ahmad Z., Ashfaq U. A. Molecular Docking Based Screening of Plant Flavonoids as Dengue NSI inhibitors. Bioinformation. 2014; 10:460-465. doi: 10.6026/97320630010460.
40. Rehman S., Ijaz B., Fatima N., Muhammad S. A., Riazuddin S. Therapeutic potential of Taraxacum officinale against HCV NS5B polymerase: In-vitro and In silico study. Biomed. Pharmacother. 2016; 83:881-891. doi: 10.1016/j.biopha.2016.08.002
44. Moghaddam E., Teoh B. T., Sam S. S., Lani R., Hassandarvish P., Chik Z., Yueh A., Abubakar S., Zandi K. Baicalin, a metabolite of baicalein with antiviral activity against dengue virus. Sci. Rep. 2014; 5:5452-5459. doi: 10.1038/srep05452.
40. Rehman S., Ijaz B., Fatima N., Muhammad S. A., Riazuddin S. Therapeutic potential of Taraxacum officinale against HCV NS5B polymerase: In-vitro and In silico study. Biomed. Pharmacother. 2016; 83:881-891. doi: 10.1016/j.biopha.2016.08.002
41. Khachatoorian R., Arunugaswani V., Raychauduri S., Yeh G. K., Maloney E. M., Wang J., Dasgupta A., French S:W. Divergent antiviral effects of bioflavonoids on the hepatitis C virus life cycle. Virology. 2012; 433:346-355. doi: 10.1016/j.virol.2012.08.029.
32. Xu J. J., Wu X., Li M. M., Li G. Q., Yang Y. T., Luo H. J., Huang W. H., Chung H. Y., Ye W. C., Wang G. C., et al. Antiviral activity of polymethoxylated flavones from Guangchenpi, the edible and medicinal pericarps of Citrus reticulata “Chachi” J. Agric. Food Chem. 2014; 62:2182-2189. doi: 10.1021/jf404310y.
27. Kehinde I., Ramharack P., Nlooto M., Gordon M. The pharmacokinetie properties of HIV-1 protease inhibitors: A computational perspective on herbal phytochemicals. Heliyon. 2019; 5:e02565. doi: 10.1016/j.heliyon.2019.e02565.
39. Ahmed-Belkacem A., Guichou J. F., Brillet R., Ahnou N., Hernandez E., Pallier C., Pawlotsky J. M. Inhibition of RNA binding to hepatitis C virus RNA dependent RNA polymerase: A new mechanism for antiviral intervention. Nucleic Acid Res. 2014; 42:9399-9409. doi: 10.1093/nar/gku632.

1. A nasal spray formulation for the prevention of contracting Coronavirus comprising of phenolic compounds including ingredients selected from a group consisting of: a water soluble bee pollen, a water soluble bee propolis, a honey, preferably Manuka honey, more preferably Manuka honey with a UMF of 20+.

2. The nasal spray formulation of claim 1, characterized in that the nasal spray formulation comprises the bee propolis and the honey; or the water soluble bee pollen, the water soluble bee propolis and the honey; suspended in a chitosan based hydrogen, and or nano-chitosan hydrogen, preferably, the nasal spray formulation comprises approximately from 0.5% to 0.10% by weight of bee pollen, 0.4% to 0.7% by weight of bee propolis, preferably 0.5% by weight of Manuka honey with a UMF of 20+, about 0.3% to 0.4% by weight of vitamin C (absorbic acid), and 0.3 zinc in an able liquid carrier.

3. The nasal spray formulation is characterized in that the bee propolis is; extracted using an extraction process that maintains the potency of the naturally occurring chemicals found in the bee propolis, preferably the extraction process is selected from the group consisting of aqueous extraction or ethanol extraction process, vacuum resistive heating extraction (VRHE) process.

4. The nasal spray formulation is characterized in that the bee propolis is an aqueous dispersion of bee propolis, preferably a water soluble bee propolis, having the key ingredient Quercetin and Rutin and vitexin, etc, that is separated via chromatography separation and enhanced by sub-micron ultra sonication via aqueous dispersion preferably, the ultra-sonication for approximately 20-30 minutes is mixed with small amounts of the water soluble bee pollen and/propolis that has not been subject to the ultra sonication for a synergistic effect, creating rapid antiviral resistance upon administration of the antiviral nasal spray.

5. The nasal spray formulation is characterized in that the nasal spray further comprises of a chitosan based hydrogel and/a nano-chitosan-based hydrogel; preferably, the nasal spray further comprises a nano-chitosan-based hydrogel and the Manuka honey; more preferably, the nasal spray further comprises a nano-chitosan-based hydrogel and the Manuka honey with a UMF of 20+.

6. The nasal spray formulation is characterized in that the bee pollen is present in an amount from 3% to 8% by weight; and/or the bee propolis is present in an amount from 0.1% to 0.8% by weight, preferably from approximately 0.1% to 0.5% by weight, and more preferably 0.3% to 0.4% by weight; and/or the honey is present in an amount from 0.4% to 0.7% by weight, preferably 0.5% by weight.

7. The nasal spray formulation is characterized in that the nasal spray formulation further comprises at least one additional bioactive substance, preferably the additional bioactive substance is one or more antioxidants and/or one or more phytochemicals and/or one or more amino acids and/or one or more proteins and/or one or more lipids, preferably one or more antioxidants and/or one or more phytochemicals, more preferably one or more antioxidants.

8. The nasal spray formulation, is characterized in that the phytochemicals are flavonoids; wherein the flavonoids are preferably selected from the group consisting of flavonoids flavone, isoflavone, flavan, isoflavan, flavanone, flavonol, flavan3-ol, dihydroflavonol, flavanonol, anthocyanidin, proanthocyanidin, aurone, chalcone, dihydrochalcone, flavonolignans, and derivatives thereof, more preferably flavones, apigenin, vitexin, quercetin, rutin, naringenin, and combinations thereof, and/or the antioxidants are one or more of a vitamin or vitamin derivative, preferably one or more antioxidants are selected from the group consisting of vitamin C, β-carotene, vitamin A, zinc, vitamin A ester, vitamin E, vitamin E ester, and phenolic compounds, more preferably vitamins C and E.

9. A method for manufacturing the nasal spray formulation of any one of the claims characterized in that comprising: a bee pollen and/or a bee propolis and/or a honey is mixed with a solvent; preferably the bee propolis is diluted with the solvent to provide a liquid mixture, and the honey with a UMF of 20+ is added to the liquid mixture; more preferably the bee propolis is diluted with the solvent and at least one additional bioactive substance to provide a liquid mixture, and the honey with a UMF of 20+ is added to the liquid mixture. Purified water is a preferred able liquid carrier. Water soluble bee propolis and bee pollen is the preferred option. Solvents should be minimised.

10. Use of the nasal spray formulation of any one of claims 1 to 10 in the preparation of medicaments is for the prevention of contracting Coronavirus and can be used as an effective treatment to minimise severity of symptoms.
